(19) United States
(12) Patent Application Publication
(10) Pub. No.: US 2013/0013066 A1
(43) Pub. Date: Jan. 10, 2013

(54) Methods and Devices for Joint Load Control During Healing of Joint Tissue

(75) Inventors: Michael E. Landry, Austin, TX (US); Anton G. Clifford, Mountain View, CA (US); Anthony J. Robins, Bellevue, WA (US); David Lowe, Redwood City, CA (US); Stefan M. Gabriel, Mattapoisett, MA (US)

(73) Assignee: Moximed, Inc., Hayward, CA (US)

(21) Appl. No.: 13/495,428

(22) Filed: Jun. 13, 2012

Related U.S. Application Data
(60) Provisional application No. 61/504,891, filed on Jul. 6, 2011.

(51) Int. Cl. A61F 2/08

(52) U.S. Cl. 623/14.12

(57) ABSTRACT

Various methods for treating a joint are disclosed herein. According to one method, a joint is surgically treated by performing a surgical repair treatment on tissue within the joint capsule; implanting a load reducing device at the joint and entirely outside of the joint capsule to reduce load transmitted by the treated tissue to allow for the tissue within the joint capsule to heal; and partially unloading the joint during healing of the surgical repair site.

### CROSS REFERENCES TO RELATED APPLICATIONS

This application claims the benefit of U.S. application Ser. No. 61/504,891, filed Jul. 6, 2011, the entire disclosure of which is expressly incorporated herein.

### BACKGROUND

Joint replacement is one of the most common and successful operations in modern orthopaedic surgery. It consists of replacing painful, arthritic, worn or diseased parts of a joint with artificial surfaces shaped in such a way as to allow joint movement. Osteoarthritis is a common diagnosis leading to joint replacement. Such joint replacement procedures are a last resort treatment as they are highly invasive and require substantial periods of recovery. Other less invasive procedures are available to repair or regrow damaged cartilage and bone of joints.

While various surgical procedures known in the art are useful in repairing damaged joint tissue and alleviating pain, there is the potential for overuse of the repaired joint. Overuse of the repaired joint may cause one or more areas of the joint to fail or become further damaged, which may require additional procedures. Depending upon the amount of remaining joint tissue, subsequent surgical procedures may be more invasive and extreme. Additionally, if the joint is overused, there may not be sufficient time for slow-healing tissue to heal within the joint.

For optimal pain relief, a repaired joint should not be fully loaded during the healing process. Both cartilage and bone are living tissues that respond and adapt to the loads they experience. Within a nominal range of loading, bone and cartilage remain healthy and viable. If the load falls below the nominal range for extended periods of time, bone and cartilage can become softer and weaker (atrophy). If the load rises above the nominal level for extended periods of time, bone can become stiffer and stronger (hypertrophy). Osteoarthritis or breakdown of cartilage due to wear and tear can also result from overloading. When cartilage breaks down, the bones rub together and cause further damage and pain. Finally, if the load rises too high, then abrupt failure of bone, cartilage and other tissues can result.

The treatment of osteoarthritis and other bone and cartilage conditions is severely hampered when a surgeon is not able to control and prescribe the levels of joint load. Furthermore, bone healing research has shown that some mechanical stimulation can enhance the healing response and it is likely that the optimum regime for a cartilage/bone graft or construct will involve different levels of load over time, e.g. during a particular treatment schedule. Thus, there is a need for devices which facilitate the control of load on a joint undergoing treatment or therapy, to thereby enable use of the joint within a healthy loading zone.

The present disclosure addresses these and other needs.

### SUMMARY OF THE DISCLOSURE

Briefly and in general terms, the present disclosure is directed towards various methods for treating a joint. Generally, a surgical procedure is performed on a joint to repair damage within the joint. These surgical procedures may be minimally-invasive or invasive. Exemplary surgical treatments include, but are not limited to, arthroscopic procedures, osteotomies, allotransplants, stem cell stimulation therapies, arthroplasties, arthrodeses, or autologous chondrocyte implantations.

As an adjunct to the surgical procedure, one or more load reducing apparatuses are also surgically implanted around the joint but outside the joint capsule. Depending upon the surgical procedure, the load reducing apparatus may be implanted prior to, during, or after the surgical procedure. The load reducing apparatus generally includes a first attachment structure configured to be attached to a first member of the joint and a second attachment structure configured to be attached to a second member of the joint. The load reducing device also includes a load absorber attached to the first attachment structure and second attachment structure, wherein the load absorber changes the load manipulating characteristics of the load reducing device.

The combination of the surgical procedure and the implantation of the load reducing apparatus allows a patient to use the joint without causing any additional damage to the repaired joint. The load reducing apparatus not only allows the joint tissue to heal but also allows for proper tissue remodeling so that biomechanically robust tissue may be formed.

According to one method, a joint is surgically treated by performing a surgical repair treatment on tissue within the joint capsule, implanting a load reducing device at the joint and entirely outside of the joint capsule to reduce load transmitted by the treated tissue to allow for the tissue within the joint capsule to heal, and at least partially unloading the joint during healing of the surgical repair site.

In another method, a joint is surgically treated by performing autologous chondrocyte implantation, implanting a load reducing device at the joint and entirely outside of the joint capsule to reduce load transmitted by the treated tissue on the chondrocyte implantation site, and allowing the new cartilage at the chondrocyte implantation site to mature for at least 6 months with reduced load bearing at the joint.

Other features and advantages of the present disclosure will become apparent from the following detailed description, taken in conjunction with the accompanying drawings, which illustrate, by way of example, the principles of the invention.

### BRIEF DESCRIPTION OF THE DRAWINGS

FIG. 1 is a side view, depicting an load reducing system attached across a knee joint;

FIG. 2 is an enlarged side view, depicting the system of FIG. 1;

FIG. 3 is a side view, depicting an another embodiment of an load reducing system having a single spring;

FIG. 4 is a side view depicting the system of FIG. 3 with the system in a position corresponding to the joint in partial flexion;

FIG. 5 is a side view of another load reducing system designed to be attached across a knee joint with a portion of the system located external to the skin;

FIG. 6 is a schematic diagram illustrating one method of treating a joint;

FIG. 7 is a schematic diagram illustrating another method of treating a joint;

FIGS. 8 and 9 are graphs shown the unloading profile pre and post surgery for two examples of load reducing systems; and

FIGS. 10 and 11 are graphs of two examples of the cellular status of joint tissue before and after surgery.

### DETAILED DESCRIPTION

Referring now to the drawings, which are provided by way of example and not limitation, the disclosed embodiments are directed towards apparatuses and methods for treating a joint such as, but not limited to, the knee joint. However, these embodiments may also be used in treating other body joints, and to alleviate pain associated with the function of diseased or misaligned members forming a body joint without limiting the range of motion of the joint.

Articular cartilage is composed basically of matrix material, water and chondrocytes. It is thought that the chondrocytes are primarily responsible for cartilage formation and vitality. Chondrocytes are sensitive to loads (both impact and cyclic) and overloading leads to cell death. Many surgical treatments for repair of joints rely on chondrocyte growth or generation, however, overloading counteracts this chondrocyte growth. Implantable joint unloading, load reducing, or load control devices can be used with a surgical repair joint treatment to improve the outcome of the primary repair treatment. Although external unloading braces are available and could be used to unload a joint during the healing process, these external unloading braces are cumbersome and thus, patient use of the devices is limited.

According to one method of the invention, a surgical procedure is performed on a joint with the aim to repair damaged joint tissue. An implantable load reducing apparatus is implanted at the joint and entirely outside of the joint capsule during or after the surgical procedure. The load reducing apparatus allows the patient to use the joint while also protecting the joint tissue by reducing the load on the joint and allowing the joint tissue to heal. Often the patient feels pain relief from a surgical intervention at a joint as soon as a few weeks after surgery. Although the patient feels pain relief, the tissue is not yet healed sufficiently to safely accommodate full weight bearing. The load reducing device is particularly important when the patient begins to bear weight on a partially healed joint. The load reducing apparatus is used to shield the healing tissue from potential overloading conditions during the time that the tissue is healing. Additionally, the load reducing apparatus can provide further pain relief as compared to only performing the surgical procedure or implanting the load reducing apparatus and in some cases can remain implanted and activated indefinitely.

Newly repaired or “immature” tissues have a different structure than mature tissue and immature tissues are not capable of supporting normal loads to the same extent as mature tissues. Overloaded immature tissue is never able to heal properly because of continuous damage caused by the overloading. The unloading device will allow the maturation to occur by reducing the load on this healing tissue.

The load reducing device may be inserted temporarily for a time period of from a few months to a few years. The load reducing device allows the target tissue (e.g., bone or cartilage) treated by the surgical procedure to fully heal and also allows for proper remodeling so that the tissue can form biomechanically robust tissue. After complete healing of the tissue, the load reducing device or a portion thereof may be removed or deactivated. Alternatively, the load reducing device can remain in place to reduce the load on the repaired joint long term, particularly for high activity patients or for heavy weight patients who may have a tendency toward reinjury of the joint.

In the adjunctive methods disclosed herein, various load reducing apparatuses may be used in conjunction with various surgical repair procedures. The surgical treatments include, but are not limited to, arthroscopic procedures, high tibial osteotomy, distal femoral osteotomy, allografts, autografts, stem cell stimulation therapies (e.g., Pridie drilling or microfracture), arthroplasty (e.g., unicondylar knee and total knee arthroplasty), or autologous chondrocyte implantation.

According to one method of treating a joint, the load reducing device may be used in conjunction with arthroscopic treatments. These arthroscopic treatments are minimally-invasive procedures in which small incisions are made around the joint for inserting a camera and other surgical tools for performing the procedure. The arthroscopic procedure may involve removing or repairing tissue. One such arthroscopic method is an arthroscopic lavage, a procedure in which blood, fluids, or loose debris are washed out of the joint. In another method, the arthroscopic treatment is arthroscopic shaving. In yet another method, the arthroscopic treatment is arthroscopic debridement. In this procedure, loose tissue (e.g. cartilage, inflamed tissue, or bone spurs) within the joint cavity is removed from the joint. In another treatment called meniscus repair a torn segment of the meniscus is removed and/or the torn edges are sutured together. In each of these procedures, the load reducing device is implanted to reduce the load on the treated tissue while the tissue heals.

In yet another method, the load reducing device may be used in conjunction with allograft, autograft, or xenograft procedures. An allograft procedure is the transplantation of cells, tissue, or organs from one individual of the same species to another individual such that there is no antigenic interaction. By way of example and not of limitation, bone, ligaments or tendons may be transplanted from a donor into a patient in an allograft procedure. In an autograft procedure, the patient's own tissue from one part of the body is used for transplantation to another part of the body. In a xenograft procedure, tissue from another species is used in the transplantation procedure. In the various allograft transplant procedures, the grafts may be large single grafts or a plurality of small grafts (mosiacplasty). The load reducing device is particularly useful for transplant procedures (either allograft, autograft or xenograft) in which load bearing cartilage or bone of the joint has been repaired.

In another method, the load reducing device is used in conjunction with stem cell stimulation therapies. One stem cell stimulation therapy is the Pridie procedure in which holes are drilled through the damaged cartilage areas of the knee into the underlying bone marrow which allows the bone marrow cells (i.e., stem cells) to grow into the damaged area of the knee. Since the bone marrow cells are stem cells (i.e., the cells are undifferentiated), the stem cells can change (i.e., differentiate) into the appropriate cells for the area in which they are growing. Accordingly, the stem cells growing in the damaged cartilage areas of the knee become cartilage cells. As an alternative to the Pridie procedure, a microfracture procedure may be performed. In a microfracture procedure, fractures are created in the bone underlying the articular cartilage by using an awl. The fractures allow blood and bone marrow (continuing stem cells) to form a clot on the damaged articular cartilage. The stem cells then differentiate and form cartilage.

In another method, the load reducing device is used in conjunction with autologous chondrocyte implantation (ACI). In ACI, a biopsy of healthy articular cartilage is removed from a patient. The harvested cartilage is then processed to obtain chondrocyte cells. These cells are grown in culture to form more chondrocyte cells. Products such as Carticel®, ChindroCelect or Hyalograft-C may be used to culture the harvested chondrocyte cells. Once there are a sufficient number of chondrocyte cells, the cells are implanted into the patient. During this surgical procedure, the damaged cartilage is removed and the surrounding cartilage is smoothed. Next, in one method, a piece of periosteum is sewn over the area absent any cartilage. The chondrocyte cells are then injected under the periosteum. The chondrocyte cells are allowed to grow and eventually form hyaline or hyaline-like cartilage.

Alternatively, in another method, the harvested chondrocyte cells are cultured with a collagen matrix. The combination of the cultured chondrocyte cells and the collagen matrix is then implanted in the area where damaged cartilage has been removed or where cartilage has been entirely worn away. This culture plus matrix combination may be secured to the defective area with fibrin glue.

In yet another method, the harvested chondrocyte cells are cultured on a three-dimensional (3-D) scaffolding. In one embodiment, the 3-D scaffolding is an alginate/agarose hydrogel. In another embodiment, the 3-D scaffolding is a type II collagen matrix. In alternate embodiments, other 3-D scaffolding materials may be used in combination with the chondrocyte cells to form a 3-D matrix, which is subsequently implanted in the patient at the site of denuded cartilage in a joint. In other embodiments, chondrocyte amplification is combined with one of the matrix systems described. The chondrocytes may mature in vitro or in vivo.

In another embodiment, the chondrocyte cells are substituted with stem cells. The stem cells are cultured, such as on a 3-D scaffold. The stem cells are allowed to differentiate and amplify in culture. Once a sufficient number of stem cells has been produced and differentiated, the 3-D scaffolding and the differentiated cells are implanted in the patient.

According to another method, the load reducing device may be used in conjunction with osteotomy procedures in which bones are surgically cut to improve joint alignment. A misalignment due to injury or disease in a joint relative to the direction of load can result in an imbalance of forces and result in cartilage degeneration and pain in the affected joint. The goal of osteotomy is to surgically realign the bones at a joint and thereby relieve pain by equalizing forces across the joint. This can also increase the lifespan of the joint. When addressing osteoarthritis in the knee joint, osteotomy involves surgical re-alignment of the joint by cutting and reattaching part of one of the bones at the knee to change the joint alignment, and this procedure is often used in younger, more active or heavier patients. The most common knee osteotomy procedure is high tibial osteotomy (HTO) which involves the surgical cutting and re-alignment of the upper end of the shin bone (tibia) to address knee malalignment. HTO addresses osteoarthritis and often results in a decrease in pain and improved function. Alternatively, distal femoral osteotomy (surgical re-alignment of the lower end of the femur to address knee alignment) may be done to treat degenerative valgus deformity of the knee. A valgus deformity of the knee also known as a “knock knee” condition, causes increased stress and degeneration of the lateral side of the knee joint.

In yet another method, the load reducing device is used in conjunction with arthroplasty procedures. In one method, the arthroplasty procedure is an unicondylar knee arthroplasty. Unicondylar (or unicompartmental) knee arthroplasty (UKA) is a minimally invasive procedure in which only the damaged side of the knee joint is replaced while leaving as much of the bone and tissue in the joint. Generally, a small incision is made to access the knee joint. The damaged portion of the knee joint (a portion of the articular surface and some bone) is removed, and prostheses are attached to tibial and femoral surfaces.

In another method, the arthroplasty procedure is a total knee arthroplasty (TKA). TKA is an invasive procedure in which one or more of articular surfaces of the tibial and femoral joint surfaces are replaced with prosthetics made from metal or plastics. In a TKA, the knee joint may be approached anteriorly through a medial parapatellar approach or a lateral or subvastus approach. Once accessed, soft tissues and bone spurs within the knee joint are removed. The distal portion of the femur and the proximal portion of the tibia are cut and bone is removed so that the prostheses can be implanted. The prostheses provides artificial articulating surfaces for the knee and removes all the natural articulating surfaces of the joint. Additionally, proper alignment of the prostheses is necessary so that the ligaments around the knee are balanced and to prevent alteration of patella height so that proper patellofemoral mechanics are maintained.

The load reducing devices described and shown herein can be used as an adjunct to the above-described or other surgical procedures performed on the tissues of a joint. The load reducing devices can provide temporary offloading to the joint tissues while the joint tissues are given the time to fully recover from surgery, to recover from another event or allow the tissue to mature into biomedically robust tissue that can withstand the force applied to the joint during normal activity or even high impact activity. The load reducing devices are preferably implanted entirely outside of the joint capsule by securing to the bones on opposite sides of the joint and traversing the joint outside of the joint capsule. The load reducing devices reduce the weight (or load) borne by the joint by partially unloading the joint and allowing some of the forces on the joint to be transmitted through the load reducing device instead of the joint tissue.

The embodiments of the load reducing devices described herein include fully implantable load reducing devices and external load reducing devices attached to the bones of the joint by implanted transcutaneous screws or pins. Some embodiments include a load reducing device including a dual spring member and other embodiments include the use of a single spring member. Although springs are shown for providing unloading, the term spring is intended to include both traditional springs, such as the coil springs shown, as well as other elements which can provide a biasing force, such a resilient materials.

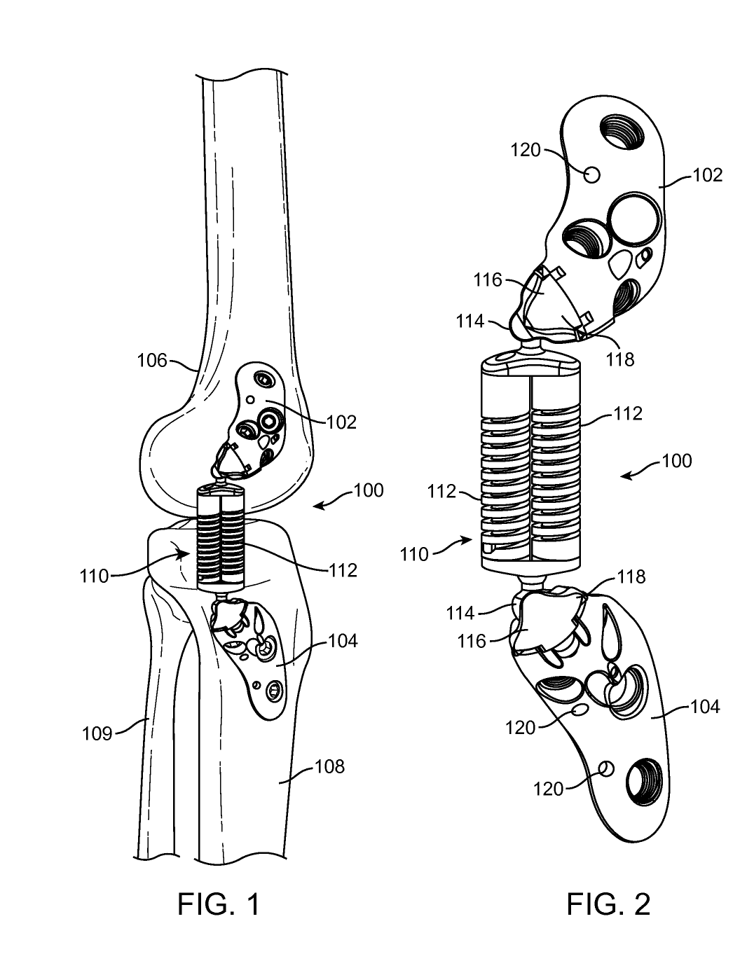

Referring now to FIGS. 1A-1C, one embodiment of a fully implantable, extra-capsular, load reducing system 100 is shown. The system includes proximal 102 and distal 104 bases positioned upon first 106 and second 108 bones, respectively of a typical body joint. This as well as the other described devices are intended to be implanted subcutaneously and entirely outside the articulating surface of a joint. As shown, the load reducing device 100 is positioned across a knee joint. However, it is to be appreciated that the load reducing devices described herein can be employed to treat other areas of a patient's body.

Conventional surgical or minimally invasive approaches can be taken to gain access to the body joint or other anatomy requiring attention. Arthroscopic approaches are contemplated when reasonable to both implant the energy manipulation assembly 100 as well as to perform one or more of the other surgical procedures described above for treating the joint. The surgical procedure to implant the load reducing system 100 is preferably performed at the same time as the surgical treatment on the joint tissue. However, the implantation of the load reducing device 100 can also be performed before or after the surgical treatment of the joint.

FIG. 1 illustrates one embodiment of an extra-articular implantable mechanical load reducing system 100 as implanted at a knee joint to unload or reduce the forces on the tissues of the medial knee joint after surgical treatment of the knee. The mechanical load reducing system 100 includes femoral and tibial bases 102, 104, respectively. An articulated absorber 110 is connected to both the femoral and tibial bases 102, 104. As shown in FIG. 1, the knee joint is formed at the junction of the femur 106, the tibia 108 and the fibula 109. Through the connections provided by the bases 102, 104, the absorber assembly 110 of the load reducing system 100 can function to absorb and reduce load on the knee joint. The system 100 is placed beneath the skin and outside the joint and resides at the medial aspect of the knee in the subcutaneous tissue. The system 100 requires no bone, cartilage or ligament resection. The only bone removal being the drilling of holes for the screws which quickly heal if screws are removed. Thus, the system 100 can be either a temporary or a permanent implant for unloading or controlling the load on the joint.

It is also to be recognized that the placement of the bases 102, 104 on the bones without interfering with the articular surfaces of the joint is made such that further procedures, such as a total knee arthroplasty (TKA), unicompartmental knee arthroplasty (UKA) or other arthroplasty procedure, can be conducted at the joint at a later date. For the later procedure, the bases 102, 104 can remain in place after removing the absorber assembly 110 or both the absorber assembly and bases can be removed. Additionally, the absorber assembly 110 can be changed out with a new absorber assembly without having to replace the bases.

Turning now to FIG. 1, it can be appreciated that the femoral and tibial bases 102, 104 include various surfaces which are curved to substantially match the surfaces of bones to which they are affixed. Moreover, various affixating structures, such as screws, are contemplated for affixing the bases 102, 104 to body anatomy.

With reference to FIG. 1, a femoral base 102 fixed to a medial surface of a femur 106 is illustrated. It is to be recognized, however, that the base 102 can be configured to be fixed to a lateral side of the femur 106 or other anatomy of the body. The proximal end of the outer surface of the femoral base 102 may reside under the vastus medialis and is designed to allow the vastus medialis muscle to glide over the outer surface of the base. The femoral base 102 is intended to be positioned on the femur at a preset location such that a center of rotation of the ball and socket joint of the absorber assembly 110 is at a particular location with respect to the center of knee rotation. According to one embodiment, the base 102 is mounted to the medial epicondyle of the femur 106 so that a femoral pivot point of the system is located anterior and/or superior to the center of rotation of the knee. Mounting the absorber 110 at this location allows the extra-articular mechanical load reducing system 100 to reduce forces during the “stance” or weight bearing phase of gait between heal strike and toe-off where forces are at their highest. Alternatively, the femoral base may be mounted at different positions on the femur to reduce forces during different phases of a person's gait.

As shown in FIG. 2, the femoral base 102 is generally elongate and includes a first curved end and a second squared mounting end which is raised to suspend the absorber 110 off the bone surface to avoid contact between the absorber and the knee capsule and associated structures of the knee joint. It is contemplated that the absorber 110 be offset approximately 2-15 mm from the surface of the joint capsule. Thus, the system 100 is extra-articular or outside of the capsular structure of the knee. Accordingly, the base 102 allows for positioning of an extra-articular device on the knee joint while preserving the knee structures including the anterior cruciate ligament (ACL), posterior cruciate ligament (PCL), Pes anserius tendon, and allowing future surgical procedures such as TKA or UKA.

As best seen in FIGS. 1-2, the squared off second ends of the femoral 102 and tibial 104 bases are shaped to mate with socket structures 118. In one approach, the sockets 118 each include a post which is press fit into a corresponding bore formed in the squared off ends of the bases 102, 104.

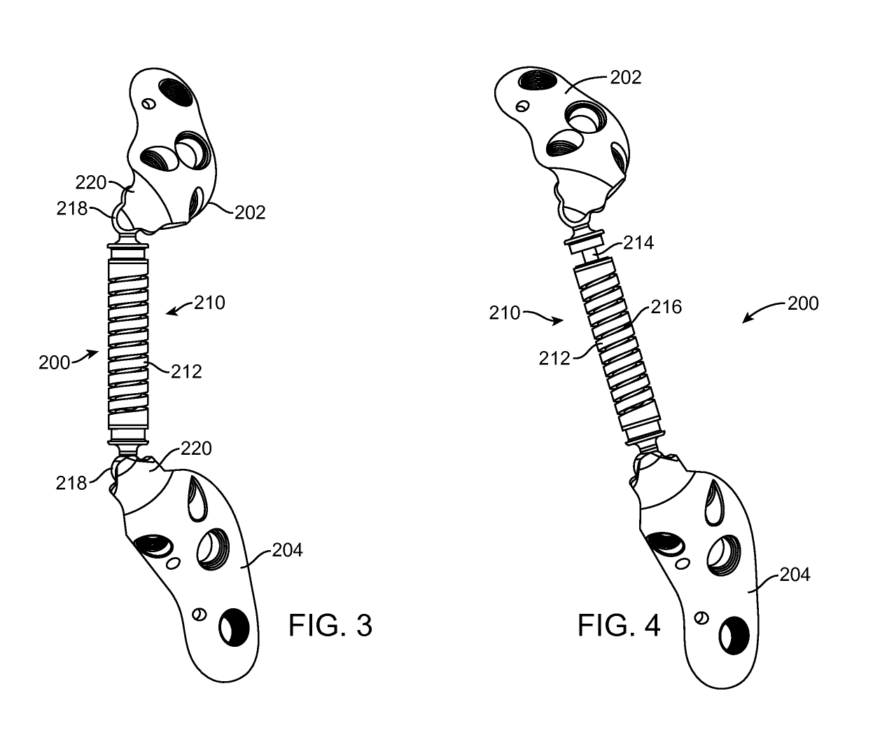

Although the bases 102, 104 are shown to be connected to the load reducing device 110 by mounts 118, the mounts may also be formed as an integral part of the base as will be shown with respect to the embodiment of FIGS. 3-4. The outer surface of the base has a low-profile and is curved to eliminate any edges or surfaces that may damage surrounding tissue when the base is affixed to bone. The inner surfaces and outer surfaces of the bases 102, 104 are not coplanar and serve differing functions which the inner surface conforming to the bone shape and the outer surface providing a smooth transition between the bone and the absorber assembly 110.

Although two compression springs 112 are shown in the load reducing device 110, one or more springs may be used. The configuration of the springs may be varied to minimize device size while maximizing its load reducing capabilities. Moreover, various types of springs such as coaxial or leaf springs can be employed and the spring structure can be placed serially and adjusted one by one.

The femoral and tibial bases 102, 104 include a plurality of openings that are sized to receive fastening members used to permanently secure the base to the bone. The openings define through-holes that may receive fastening members such as compression screws and/or locking screws. The openings may have divergent bore trajectories to further maximize the pull forces required to remove the base from the bone. Although divergent bore trajectories are shown, converging trajectories may also be used as long as interference between the screws is avoided. The number and trajectories of the openings may be varied in alternate embodiments.

The various load reducing devices in the present application are shown without a protective covering or sheath but it is contemplated that they can be within a protective covering or sheath to protect the moving elements from impingement by surrounding tissues and to prevent the devices from damaging surrounding tissue. The bases 102, 104 may be provided with attachment means such as holes for receiving a fastener to attach the sheath to the bases. Examples of protective coverings are described in U.S. Patent Application Publication Nos. 2009/0275945 and 2009/0276044 which are incorporated herein by reference in their entirety.

Although the use of compression screws are described herein, the methods and systems described can be employed without the use of a compression screw and may use the alternative of an instrument designed for delivering compression while locking screws are placed.

In certain embodiments, the load transferred from the absorber to the base can change over time. For example, when the base is initially fixed to the bone, the fastening members carry all the load. Over time, as the base may become osteointegrated with the underlying bone at which time both the fastening members and the osteointegrated surface carry the load from the implanted system. The loading of the bases also varies throughout motion of the joint as a function of the flexion angle and based on patient activity.

The inner surfaces of the femoral and tibial bases 102, 104 can be roughened or etched to improve osteointegration. Alternatively, the inner surface bone contacting surfaces can be modified in other ways to induce bone growth. In one example, the inner surfaces may be coated with bone morphogenic protein 2 (BMP-2), hydroxyapatite (HA), titanium, cobalt chrome beads, any other osteo-generating substance or a combination of two or more coatings. According to one embodiment, a titanium plasma spray coating having a thickness of approximately 0.025 in.±0.005 in. is applied to the inner bone contacting surface 1470. In another embodiment, a HA plasma spray having a thickness of approximately 35 μm±10 μm is applied to facilitate osteointegration. The portions of the inner surfaces of the bases which are not in contact with the bone including the curved offset surfaces of the bases may or may not be treated in the same manner to improve osteointegration at the bone contacting surface.

It is contemplated that femoral and tibial bases 102, 104 can be provided in two or more versions to accommodate patient anatomies. The two or more versions of the bases 102, 104 form a set of bases of different shapes and/or sizes which are modular in that any one of these bases can be used with the same absorber. The bases are described in further detail in U.S. patent application Ser. No. 12/755,335 entitled “Femoral and Tibial Bases” which is incorporated herein by reference in its entirety.

The implantable load reducing systems 100 described herein have a total of 8 degrees of freedom including two universal joints each having three degrees of freedom and the absorber having two degrees of freedom (translation and rotation). However, other combinations of joints may be used to form an implantable load reducing system, such as a system having 5, 6 or 7 degrees of freedom.

The figures have illustrated the implantable mechanical load reducing systems designed for placement on the medial side of the left knee. It is to be appreciated that a mirror image of the femoral base 102 would be fixable to the medial surface of the right femur for the purposes of unloading or reducing a load on the medial compartment of the knee. In an alternate embodiment, the femoral and tibial bases 102, 104 and the absorber 110 may be configured to be fixed to the lateral surfaces of the left or right knee joint and to reduce loads on the lateral compartment of the knee or of other joints.

FIG. 1 shows the knee joint at full extension with load being applied to the two springs 112 of the load reducing device. When the knee joint is flexed the load reducing device 110 extends and zero load is applied to the springs by virtue of the springs 112 being shorter than the length of the piston shafts on which the springs are mounted. The load reducing device lengthens as the knee swings from full extension to flexion and subsequently shortens as the knee swings from flexion to full extension such that the springs begin to be compressed between the ends of the device to absorb the load that the knee articulating surfaces normally would experience. The load reducing device 110 and bases 102, 104 are mounted at the joint such that, the articulating surfaces of the knee then contact one another and carry the load in combination with the load reducing device. Accordingly, the load reducing device and the natural joint share the total load on the joint and the springs 112 do not “bottom out.” This load reducing device is described in further detail in U.S. patent application Ser. No. 12/843,381 filed Jul. 26, 2010 and entitled “Absorber Design for Implantable Device,” which is incorporated herein by reference in its entirety. However, depending on the amount of unloading desired, the load can be carried 100 percent by the load reducing device 110 during some portion of the healing process.

While screws are used to fix the femoral and tibial bases 102, 104 to the bone, those skilled in the art will appreciate that any fastening members known or developed in the art may be used in addition to or as an alternative to screw fixation to accomplish desired affixation. Additional instruments and methods which are usable with the bases are described in detail in U.S. patent application Ser. No. 12/915,606 entitled, “Positioning Systems and Methods for Implanting an Energy Absorbing System,” which is incorporated herein by reference in its entirety.

The femoral and tibial bases 102, 104 may also include a plurality of holes 120 that may be used during alignment of the bases 102 on the femur and tibia and sized to receive structures such as a K-wire. Optionally, the bases 102, 104 may include a plurality of holes, teeth or other surface features (not shown) to promote bone in-growth thereby improving base stability. The inner bone contacting surfaces of the bases can be a roughen for improving osteointegration. Alternatively or additionally, the inner surfaces can be coated to induce bone growth.

The bases 102, 104 have a generally low-profile when mounted to the bone. The mounting ends of the bases 102, 104 which are connected to the absorber 110 are shown offset from the surface of the tibia and femur allowing the absorber to move throughout a range of motion while avoiding anatomical structures and maintaining a low profile of the base. Together the tibial and femoral bases are configured to receive the absorber in a position where the absorber plane is substantially parallel to a line connecting the medial aspects of the femoral and tibial condyles.

Referring to FIGS. 3 and 4, one embodiment of a single spring load reducing system 200 includes bases 202, 204 and an load reducing device 210 connected there between having a single spring 212. The spring 212 is mounted about telescoping arrangement including a piston 214 and an arbor 216. The piston 214 and arbor 216 are each connected to a ball 218. The balls 218 at either end of the load reducing device 210 are received in sockets of the bases 202, 204. Unlike the device of FIGS. 1 and 2, the system 200 has sockets 220 formed as integral parts of the bases 202, 204.

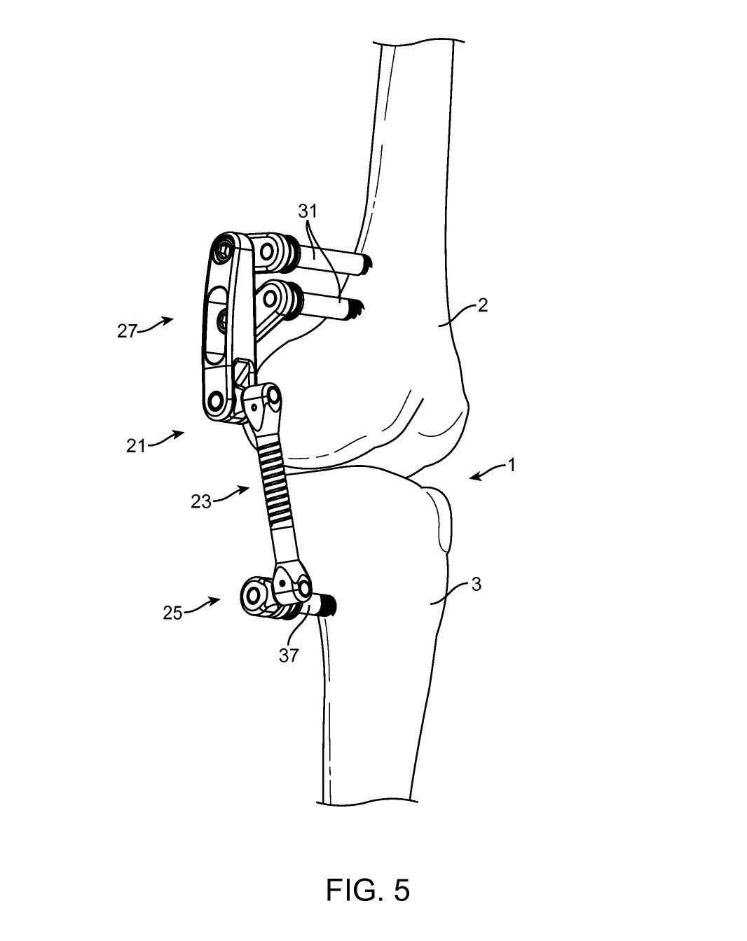

FIG. 5 illustrates a transcutaneous implantable knee load reducing device 21 according to an aspect of the invention for a knee joint 1. The knee joint 1 comprises a first member 2, which may be a femur, and a second member 3, which may be a tibia. The device 21 shown in FIG. 1 is shown as having an external component on a medial side of a left knee joint 1, but it will be appreciated that an external component of the device may be disposed on the lateral side of the joint or on both sides of the joint.

The device 21 comprises a load absorber 23 that is ordinarily entirely or at least substantially outside of the user's skin. The load absorber 23 has a first and a second mating portion 25 and 27 and a piston, spring, arbor assembly disposed between the first and the second mating portions. The device 21 further comprises a pair of first percutaneous anchors 31 and a second percutaneous anchor 37. The first and second mating portions 25 and 27 and the first and second anchors 31 and 37 are configured so that the load absorber 23 is disposed externally of a user's skin. At least the first and second anchors 31 and 37 will ordinarily have a coating, such as a TiAg coating, to reduce the possibility of infections.

The first and second mating portions 25 and 27 are easily attached to and detached from first and second anchors 31 and 37, such as by providing suitable quick-release fittings. The load absorber 23 ordinarily comprises a spring and a piston and arbor assembly. When a user applies a load to the knee joint 1, such as by standing or walking, the spring will tend to absorb some or all of the force applied to the knee joint and thereby reduce load on the knee joint. The transcutaneous load reducing device 21 of FIG. 5 is further described in U.S. Patent Application No. 61/504,886 (Attorney Docket No. 83456.0079) entitled “Transcutaneous Joint Unloading Device and Method” and filed on Jul. 6, 2011 which is incorporated herein by reference in its entirety.

The various embodiments of the bases 102, 104, 202, 204 describe herein may be made from a wide range of materials. According to one embodiment, the bases are made from metals, metal alloys, or ceramics such as, but not limited to, Titanium, stainless steel, Cobalt Chrome or combinations thereof. Alternatively, the bases are made from thermo-plastic materials such as, but not limited to, high performance polyketones including polyetheretherketone (PEEK) or a combination of thermo-plastic and other materials. Various embodiments of the bases are relatively rigid structures. Preferably, the material of the base is selected so that base stiffness approximates the bone stiffness adjacent the base to minimize stress shielding.

Biologically inert materials of various kinds can be employed in constructing the load reducing devices or load reducing devices 110, 210 of the present invention. For example, the load reducing devices can be titanium or titanium alloy, cobalt chromium alloy, ceramic, high strength plastic such as polyetheretherketone (PEEK) or other durable materials. Combinations of materials can also be used to maximize the properties of materials for different parts of the device. At the bone interface surfaces, the materials can be coated with a material which promotes osseointegration. At the wear surfaces including the ball and socket joints and the piston and arbor telescoping joints, the material may include a combination of metal-on-polymer, metal-on-metal, metal-on-ceramic or other combinations to minimize wear.

In one example, the single spring load reducing system 200 of FIGS. 3 and 4 can be formed of PEEK to provide an implant particularly suited for shorter term use, such as for unloading a joint in the period following a surgical repair procedure. The single spring system 200 can include PEEK or carbon fiber reinforced PEEK bases 202, 204 coated with a material such as an HA coating or other coating for improving osseointegration. The absorber 210 can include PEEK or reinforced PEEK arbor and piston arrangements combined with a metal spring. This material combination results in a PEEK on PEEK articulation of the ball and socket joints for improved wear properties.

The load reducing system has the capacity to absorb energy in addition to transfer energy from the joint. The energy absorption of the dual or single spring can be expressed as the product of force and displacement. Although actual springs are used to show various embodiments, these elements could also be substituted with a material or other device with spring-like characteristics (e.g., an elastomeric member, hydraulic, pneumatic, or magnetic member,). Examples of elastomers include thermoplastic polyurethanes such as Tecoflex, Tecothane, Tecoplast, Carbothene, Chronthane and ChronoFlex (grades AR, C, AL) which also could be employed as a dampener. Moreover, materials such as Pebax, C-flex, Pellathane and silicone and silicone foam can also be employed.

It is to be borne in mind that each of the disclosed various structures can be interchangeable with or substituted for other structures. Thus, aspects of each of the bending spring, cam engagement, segmented support and piston support assemblies can be employed across approaches. Moreover, the various manners of engaging load reducing structure with attachment structure and attachment structures to body anatomy can be utilized in each approach. Also, one or more of the various disclosed assemblies can be placed near a treatment site and at various angles with respect thereto. Pressure sensing and drug delivery approaches can also be implemented in each of the various disclosed embodiments.

Certain members of most embodiments of the present invention can be made in multiple parts designed for modular assembly of different sizes and shapes and for easy removal and, if necessary replacement of some members or parts of members without removal of the entire system. The permanent parts include fixation components which have bony ingrowth promoting surfaces and are responsible for fixation of the system to the skeletal structure. The removable parts can include the mobile elements of the system. Various shapes of bases are contemplated and described. Moreover, it is contemplated that various sized and similar shaped bases be made available to a physician in a kit so that a proper fit to variably sized and shaped bones can be accomplished. In that regard, it is contemplated that up to three or more different femoral and tibial bases can be available to a physician.

Although the mechanical load reducing systems 100, 200, 21 which have been illustrated as used to reduce loading on the medial knee, they may also be used in other joints such as the finger, hand, toe, spine, elbow, hip and ankle. Other base configurations and shapes which may be suitable for use in some of these applications include those disclosed in U.S. patent application Ser. No. 12/112,415 entitled “Femoral and Tibial Base Components” and U.S. patent application Ser. No. 12/755,335 entitle “Femoral and Tibial Bases” which are incorporated herein by reference in their entirety.

As stated, the above-described load reducing apparatuses can be used as an adjunctive therapy to surgical procedures used to repair a joint. The surgical treatments include, but are not limited to, arthroscopic procedures, high tibial osteotomy, distal femoral osteotomy, allografts, autografts, stem cell stimulation therapies (e.g., Pridie drilling or microfracture), arthroplasty (e.g., unicondylar knee and total knee arthroplasty), or autologous chondrocyte implantation.

Adjunctive Use—Cartilage Repair

The various load reducing apparatuses may be used as an adjunct to other treatments for joint injuries. For example, the load reducing device may be used in conjunction with surgical treatments for meniscus or other cartilage repair. The meniscus is crescent-shaped fibro-cartilage structure that is configured to transmit load from a spherical surface (femoral condyle) to a flat surface (tibial plateau). Portions (a peripheral one third) of the meniscus have a vascular supply so it is capable of healing. Accordingly, tears in about the outer third of the meniscus may be surgically repaired. As discussed above, cartilage repair relies on chondrocyte growth or generation, however, overloading counteracts this chondrocyte growth. The ability of these meniscus tears to heal completely depends on the loading that the joint experiences during the healing process. The implantation of a load reducing apparatus as described herein for a period of one to two years can assist in reducing the load on the joint and allowing the tissue to completely heal. In one example, the unloading is decreased as the healing progresses, either by gradual decrease or stepwise decrease in load reduction provided by the implanted device.

The load reducing device can also be used in connection with cartilage resurfacing of any type. In order for resurfaced cartilage to heal correctly the joint must be protected from overloading until joint surfaces have healed. In one contemplated approach, the load reducing device is removed approximately 6 to 24 months after cartilage repair surgery.

Adjunctive Use—Allograft and Autograft

In yet another method, the load reducing device may be used in conjunction with allotransplant procedures such as, but not limited to, allografts, autografts, or xenografts. An allograft procedure is the transplantation of cells, tissue, or organs from one individual of the same species to another individual such that there is no antigenic interaction. In an autograft procedure, the patient's own tissue from one part of the body is used for transplantation to another part of the body. In a xenograft procedure, tissue from another species is used in the transplantation procedure. In the various allotransplant procedures, the grafts may be large single grafts or a plurality of small grafts (mosiacplasty).

Meniscal injuries may be repaired by allograft, autograft or xenograft transplantation. Many types of allograft procedures are currently used which involve transplantation of tissue including fresh chondrocyte tissue from a tissue bank or cultured chondrocyte tissue. Allografts and autografts are utilized to treat a broad spectrum of articular and osteoarticular lesions including both focal chondral defects and established osteoarthrosis. Allograft of autograft implants are generally used in conjunction with debridement. The graft material is surgically placed at the location of the defect and is protected by the suturing of a periosteal flap, a small piece of soft tissue from the tibia, which is sutured over the damaged area to serve as a physical barrier during recovery.

Carticel is one example of an autologous cultured chondrocyte product used for the repair of cartilage defects, such as defects of the femoral condyle (medial, lateral or trochlea). Carticel is grown from the patient's own chondrocytes which are removed arthroscopically from a non load-bearing area during a first surgical procedure. The harvested cells are grown in vitro and then after a cell proliferation period, the patient undergoes a second surgery in which the cultured chondrocytes are surgically injected into the patient. These cells are held in place by a periosteal flap. The implanted chondrocytes can then divide and integrate with surrounding tissue under the flap. Other cultured chondrocyte systems are also being developed which are grown with their own or a synthetic matrix to avoid the use of the periosteal flap covering.

When used with allograft or autograft implantation, the load reducing device of the present invention is implanted, preferably at the time the cartilage graft material is placed. The load reducing device reduces the weight born by the joint following surgery to allow the cartilage graft to integrate with the surrounding tissue and create hyline type cartilage. The load reducing device remains in place and continues to unload the joint during the healing process which generally takes about 6 months to 3 years. Preferably, the device remains in place for about 2 years or longer and in some cases, the patient may choose to leave the device in permanently.

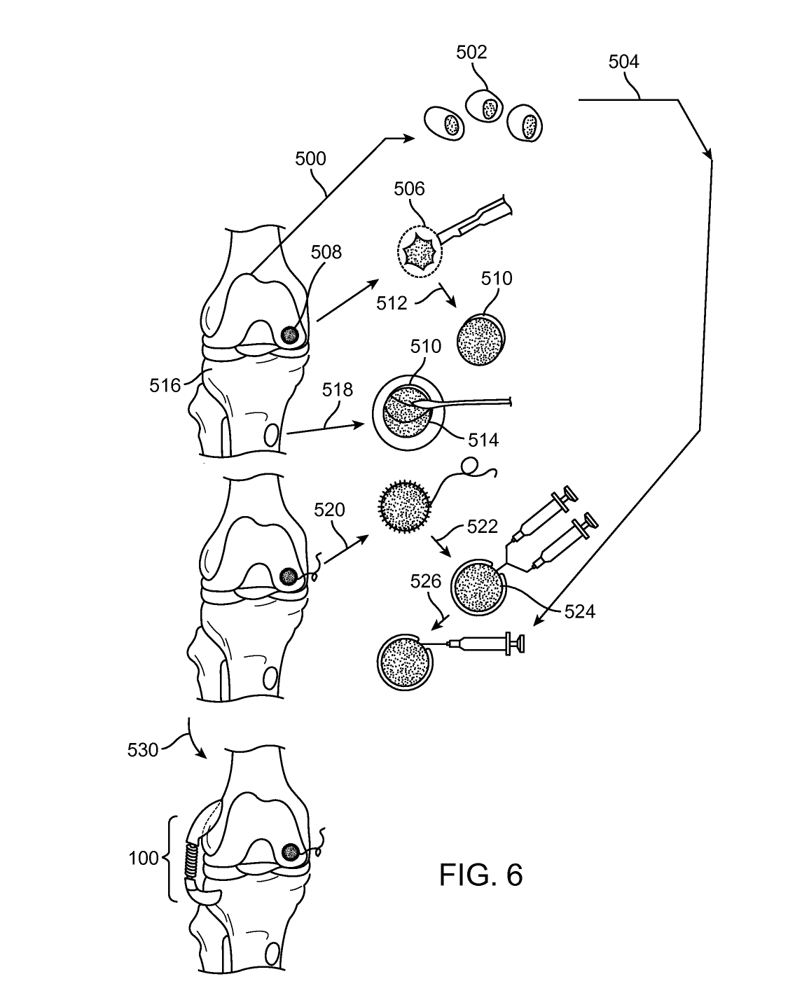

FIG. 6 illustrates one method for treating a joint using a load reducing device in conjunction with autologous chondrocyte implantation (ACI). In this method, a biopsy of healthy articular cartilage is removed from a patient at step 500. The harvested cartilage is then processed to obtain chondrocyte cells 502. These cells are grown in culture to form more chondrocyte cells at step 504. Products such as Carticel, ChondroCelect, of Hyalograft-C may be used to culture the harvested chrondocyte cells. Once there are a sufficient number of chondrocyte cells, the cells are ready for implantation into the patient. Prior to implanting the chondrocyte cells, at step 506, the dead cartilage 508 is removed. The surrounding cartilage is smoothed to form a generally uniform void 510 at step 512. A piece of periosteum 514 is taken from the tibia 516 at step 518. The piece of periosteum 514 is sewn over the void 510 at step 520. At step 522, the piece of periosteum 514 is sealed with fibrin glue 524 to prevent leakage when the chondrocyte cells are implanted. The chondrocyte cells are then injected under the periosteum at step 526. The chrondocyte cells are allowed to grow and eventually form hyaline or hyaline-like cartilage.

Alternatively, in another method, chrondocyte cells are harvested from a patient. The harvested chondrocyte cells are amplified and cultured with a collagen matrix. The combination of the cultured chondrocyte cells and the collagen matrix is then implanted in the areas lacking cartilage. This cultured combination may be secured to the defective area with fibrin glue and without the need to cover the implanted material with the periosteum.

Next, at step 530, the load reducing device 100 such as those described above is implanted into the patient during the same procedure as the implantation of the chondrocyte cells. It will be appreciated that the load reducing device may be implanted before or after the implantation of the chondrocyte cells. For example, an alternate method, the load reducing device 100 is implanted into the patient when the biopsy is taken from the patient. The load reducing device 100 can be implanted on the outside (laterally) or inside (medially) portion of the knee joint. The load reducing device 100 is positioned at the knee joint to reduce forces on the knee, but the device is implanted outside the joint cavity. In one contemplated approach, the load reducing device 100 is removed approximately 6 to 24 months after the graft procedure and load reducing device implantation.

Adjunctive Use—Biologics

Many articular cartilage biologic treatments are on the horizon including development of cytokines, growth factors, gene therapies, stem cells, and mesenchymal precursor cell therapies. The concept of harvesting stem cells or other cells and re-implanting them into one's own body to regenerate organs and tissues including cartilage has been embraced by a number of researchers. These biologic treatments are delivered locally by a surgical procedure and held in place in a manner similar to the allograft and autografts with a matrix, periosteal flap, or the like. Like the allografts or autografts, these biologic therapies can fail due to too much load being applied too soon. Patients having these biologic treatments are generally instructed to follow a rehabilitation regimen that involve minimal weight bearing for an extended period of time in order to allow the treatment heal. However, patients are often eager to get back to normal activity earlier than the suggested time period and often do not give the treatment the needed time to fully heal. These treatments can be more effective if the joint tissue is given and extended time period for healing to take place. This healing time is provided by the implantation of the load reducing device of the present invention. The load reducing device remains in place and continues to unload the joint for about 6 months to 3 years, and preferably, the device remains in place for about 2 years or longer.

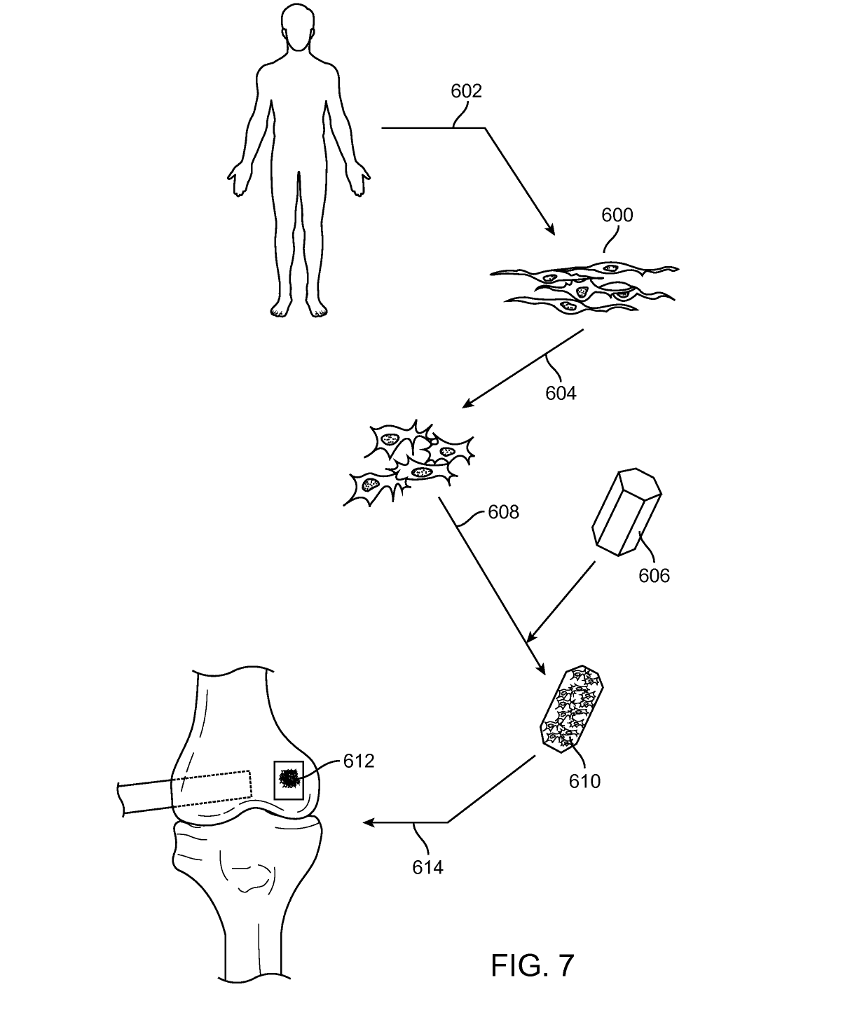

In the method of FIG. 7, undifferentiated stem cells 600 also can be obtained through lipoaspiration at step 602. The stem cells 600 are separated and amplified at step 604. The stem cells are cultured on a 3-D scaffold 606 at step 608. The stem cells are allowed to differentiate and amplify in culture. Once a sufficient number of stem cells has been produced, the 3-D scaffolding with the differentiated cells 610 are implanted into the site of the defect 612 at step 614.

In those methods using a 3-D matrix 606, the 3-D scaffolding may be a protein scaffold composed of collagen, gelatin, fibrin, or laminin. Other 3-D scaffolding compositions may be various natural materials including polysaccharides such as, but not limited to, agarose, alignate, cellulose, hyaluronic acid, or any combination thereof. Alternatively, the 3-D scaffolding may be composed of artificial materials such as, but not limited to, carbon fiber, calcium phosphate, DACRON®, polybutyric acid, polyestherurethane, polyethylmethacrylate, polyglycolic acid (PGLA), polylactic acid (PLA), or TEFLON®. In one embodiment, the 3-D scaffolding is an alginate/agarose hydrogel. In another embodiment, the 3-D scaffolding is a type II collagen matrix. The load reducing device 100 can be removed approximately 6 to 24 months, preferably approximately 12-24 months, after implantation or injection of the biologic.

Adjunctive Use—Stem Cell Stimulation Therapies, Pridie and Microfracture

In another method of treating damaged or degenerated cartilage, the load reducing device is used in conjunction with stem cell stimulation therapies. One such stem cell stimulation therapy is the Pridie procedure. In the Pridie procedure, a damaged area of articular cartilage in the knee is surgically accessed. The damaged area is debrided and then holes are drilled into the underlying bone marrow. The holes allow the bone marrow cells (i.e., stem cells) to grow into the damaged area of the knee. Since the stem cells are undifferentiated, these cells can change (i.e., differentiate) into the appropriate cells for the area in which they are growing. Accordingly, the stem cells growing in the damaged cartilage areas of the knee can differentiate into cartilage cells. Optionally, Pridie rods, which are carbon fiber tubes, may be placed within the holes. The Pridie rods keep the holes clear and provides a pathway for the stem cells to migrate from the underlying bone marrow to the articular surface of the knee.

As an alternative to the Pridie procedure, a microfracture procedure may be performed. The microfracture procedure is a minimally-invasive procedure. Small incisions are made for an arthroscope and the surgical instruments. In a microfracture procedure, the articular surfaces of the knee are accessed, and the damaged areas of the articular surfaces are cleared. Fractures are then created in the bone underlying the articular cartilage by using an awl. The fractures allow blood and bone marrow (containing stem cells) to form a clot on the area of damaged articular cartilage. Over time, the stem cells within the clot can differentiate and form cartilage. While the procedure is minimally-invasive, recovery from the procedure is difficult. The patient needs to stay off the joint for at least four weeks. Additionally, for optimal re-growth of the joint, the patient should undergo physical therapy using continuous passive motion. The patient must take care not to become too physically active on the joint (e.g., running or jumping) even though the patient does not feel any discomfort or pain. In other words, the patient is prone to overuse the joint as joint pain is typically alleviated.

Bony defects can also be repaired by seeding with biologics or stem cells or by cell stimulation methods and these repaired bone defects can be given an opportunity to heal by implantation of the load reducing devices.

When a load reducing device may be implanted at the joint but outside the joint capsule after the microfracture or other cell stimulation procedure, the chances of joint overload are reduced. The load reducing device can allow for cartilage repair to occur as the a portion of the forces on the joint are unloaded for at least a part of the patient's gait cycle. Again implantation of the load reducing device for a period of about 6 months to 3 years, and preferably about 2 years or longer should allow the mesenchymal stem cells from the bone marrow to heal and form repair tissue consisting of fibrous tissue, fibrocartilage or hyaline-like cartilage. The load reducing device 100 can be removed approximately 6 to 24 months, preferably approximately 12-24 months, after implantation or injection of the biologic.

Adjunctive Use—Osteotomy

According to another method, the load reducing device may be used in conjunction with osteotomy procedures in which bones are surgically cut to improve alignment of the bones. The goal of osteotomy is to surgically re-align the bones at a joint and thereby relieve pain by equalizing forces across the joint. This can also increase the lifespan of the joint. This procedure is often used in younger, more active or heavier patients. The use of the load reducing device in combination with an osteotomy procedure can achieve the dual goals of realigning the load bearing surfaces of the joint and reducing the load on the joint.

One osteotomy procedure is a high tibial osteotomy (HTO) in which the upper end of the shin bone (tibia) is surgically realigned. HTO is typically performed to address osteoarthritis and it often results in a decrease in pain and improved function. However, often an HTO is useful only in delaying the eventual need for a future arthroplasty procedure. The combination of an HTO surgery with the implantation of a load reducing implant can further delay and in some cases even prevent the future arthroplasty. The load reducing device 100 can be implanted at the time of the osteotomy and maintained in place permanently following the osteotomy procedure to continuously reduce the loading on the joint.

Adjunctive Use—Arthroplasty

In yet another method, the load reducing device is used in conjunction with an arthroplasty procedure. Unicondylar (or unicompartmental) knee arthroplasty or total knee arthroplasty (TKA) procedures can fail due to instability, wear or aseptic loosening. In the case of an arthroplasty which has failed for one of these reasons, when caught early, the arthroplasty can be salvaged by implanting a load reducing device which may delay the need for revision of the arthroplasty for a number of years or possibly permanently. For example, in the case of loosening, unloading with the load reducing device may give the arthroplasty implant a chance to better incorporate with the bone. Also, in the case of instability, often resulting in increased and/or uneven wear of the joint replacement device, the load reducing device can help to reduce loads/wear and preserve the joint replacement avoiding the need for a second joint replacement surgery. As the surgery to implant the load reducing device is much less invasive than the second joint replacement surgery it is preferably in many cases. In the case of the adjunctive use of the load reducing device to salvage a failed arthroplasty procedure, the load reducing device may remain implanted for the remainder of the life of the joint replacement.

In another example, the load reducing device can be implanted at the time of the arthroplasty procedure and remain implanted for a limited period of time. The implantation of the load reducing device during arthroplasty can offload the joint replacement during incorporation of the joint replacement allowing the joint replacement to heal completely under reduced loading conditions. In this case, the load reducing device can be incorporated into or attached directly onto the joint replacement. In this arrangement the load reducing device may be either temporary or permanent.

Adjustment of Load Reducing Devices

In a contemplated method, the load reducing devices 100, 200, 21 can be initially configured to eliminate or reduce loads to a desired degree, and to be later adjusted or altered as patient needs are better determined or change (i.e. as healing progresses). Accordingly, post-operative alterations are contemplated. Further, it is also contemplated that in one embodiment, there is no initial load manipulation until the interventional site heals and the device is firmly implanted while the patient is not yet load bearing. The device can provide distraction forces and carry all of the load to an extent that the joint surfaces are separated when the joint is fully load bearing. This distraction can continue for up to three months (or preferably two months) and then later the device can be adjusted to accomplish energy absorption without distraction. Moreover, as needs change, the method can involve removal or replacement of one or more components of the load reducing assembly to adjust the load supported by the load reducing assembly. The load reducing devices can be adjusted after implantation manually by external means, by neural signals of muscle activation, in a timed manner by use of absorbable materials. Examples of adjustable designs are described further in U.S. patent application Ser. No. 12/113,004 entitled “Adjustable Absorber Designs for Implantable Device” which is incorporated herein by reference in its entirety. Various approaches can be used to adjust, activate or deactivate the load reducing devices, either prior to surgery, intra-operatively or post-operatively.

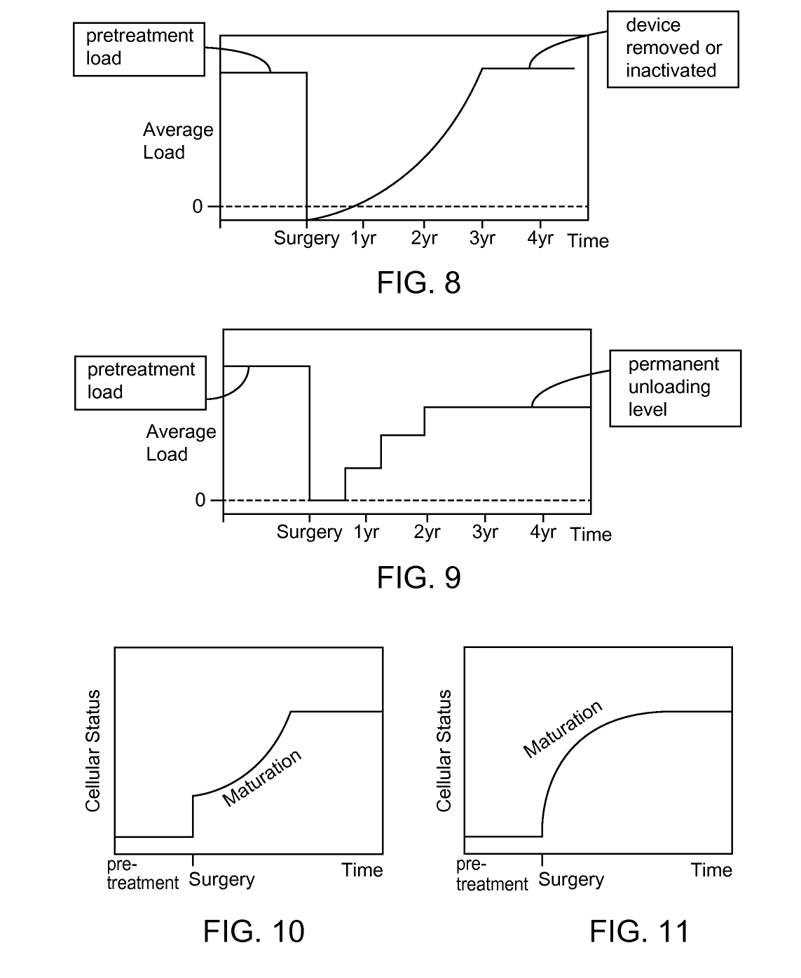

FIGS. 8 and 9 describe the unloading profile pre and post surgery for two examples, other profiles and combinations thereof are also contemplated. In FIG. 8, the pretreatment load on the joint is shown on the left side of the graph. At the time of surgery the load control device is implanted and is set to unload more than 100% of the joint load. Thus, for some initial healing period after surgery the joint is subject to distraction to facilitate regeneration tissues. At some time during the first year post surgery and preferably about 3 months or less, the complete unloading transitions into partial unloading of the joint. The curve shown in FIG. 8 representing the change in unloading over time. Although FIG. 8 shows gradual change in the early time periods with increasing change toward 3 years, other regimens can also be used including a linear change, fast initial change, or stepwise change. In the example of FIG. 8 no unloading is present after 3 years and the device can be removed or deactivated.

FIG. 9 shows an unloading of 100% at the time of surgery followed by stepwise adjustment of the unloading to levels of less unloading over the following 2 years until some low level of permanent unloading is reached.

FIGS. 10 and 11 show examples of the cellular status of the joint tissue before and after surgery. Depending on the type of treatment, there likely will be an initial improved cellular status at the time of surgery followed by a period of slow (FIG. 10) or faster(FIG. 11) maturation and recovery of the tissue.

In one embodiment of an automatically adjustable base design, the base is secured to the bone with absorbable polymer inserts in the screw holes of the base. Upon resorbtion of the polymer material, the base shifts to a second position allowing the natural joint to begin carrying a greater percentage of the weight. In another automatically adjustable absorber design, one or more absorbable polymer spacer is provided within the absorber and the spacers are resorbed after a predetermined time period resulting in less shielding over time.

The load reducing systems described herein can be made in a modular fashion to allow the ability to exchange some parts of the system due to unanticipated wear, patient condition change or newer improved systems being available. Additionally if the patient subsequently requires further surgery the entire load reducing system may be removed to facilitate the additional procedure.

It has also been contemplated that an implantable sensor unit can be configured at an interventional site to detect and keep track of indicators associated with changes in tissue density. One approach is described in WO 2007/098385, the entire contents of which are incorporated by reference. The implantable sensor unit can be configured for wireless communication with an external device and the external device can also be configured for wireless communication with the implantable sensor unit. In particular, the external device is adapted for retrieving, storing, and displaying, in human intelligible form, the tissue density data detected by the implantable sensor unit. The implantable sensor can additionally be affixed to bone of the skeletal system such that it may monitor the bone, adjacent soft tissues, such as muscles, nerves and connective tissues. The sensor may be within or integral to an artificial implant attached to the skeletal system, attached to an artificial implant, adjacent to an artificial implant, or any combination of these locations.

The implantable sensor can include a sensor, a signal processor, a memory unit, a telemetry circuit, and a power source. The sensor can be an acoustic transducer responsive to acoustic signals transmitted through human tissue. Further, it is fully contemplated that the sensor may include other electronics and components adapted for monitoring indicators of changes in tissue structure including deterioration and/or healing. The disclosed sensor has applications throughout the skeletal system including the hip, knee, ankle, elbow and jaw joints and load bearing bones such as the skull and long bones. Such disclosed sensors are useful to evaluate tissue properties and detect changes to tissue in the skeletal system. The sensor also has a particular application with respect to detecting changes in bone density as it relates to osteoporosis and the sensor can detect tissue density changes with respect to tissue around fixation implants, joint implants, or any other type of implant. Moreover, an acoustic sensor may also be used to detect changes in viscosity. Thus, the sensor may be utilized to listen for changes in bodily systems and organs and alert healthcare professionals to any impending or potential problems.

Temporary Load Reducing Devices

Most of the load reducing devices described herein can be made temporary by designing the devices for removal after healing of the joint tissues. Devices can be designed with smooth bone contacting surfaces to prevent osseointegration and allow easy surgical removal. Alternatively, the bases of the device can remain implanted with the absorber removed surgically.

In other temporary designs, portions of the implant can be biodegradable. For example, the spring can be biodegradable to gradually reduce unloading of the joint. Sequential resorbtion of the components of the implantable system can be used to form a temporary and completely resorbable device. Alternatively, one or more quick release couplings can be used to remove the load reducing device while leaving portions, such as the screws and fasteners implanted permanently.

Other Load Reducing Devices

Other load reducing devices, such as the unlinked unloading devices described in U.S. Patent Application No. 61/351,446, entitled “Unlinked Implantable Knee Unloading Device,” which is incorporated herein by reference in its entirety, can also be in connection with the present invention. In the unlinked unloading devices, the mating ends of the load reducing device are not connected together such that unloading is provided during the extension stage of the gait cycle when the mating ends contact one another and the mating portions are separated and not providing any unloading of the joint at some desired angle of flexion. Certain of the contemplated mechanisms can be made to be completely disengaged mechanically and then be brought into action under various conditions and during certain phases of the gait cycle. This discontinuous functionality—and the ability to tune that functionality to a particular patient's gait or pain is consequently a feature of the present invention. For example, by selection from a variety of different available lengths of first or second members, the unloading can be tailored to a particular portion of the gait cycle. In one embodiment, the first and second members are configured to engage one another to absorb at least a portion of the total load applied to the knee during at least 5 degrees and during no more than 60 degrees of a natural range of motion of the knee.

Additional examples of absorber structures and structures for fixing the absorber structures to bone are describe in U.S. patent application Ser. No. 12/488,260 entitled “Implantable Brace for Providing Joint Support” which is incorporated herein by reference in its entirety.

In one contemplated embodiment, the load reducing system can absorb at least a portion of a load found within a knee joint during at least five degrees and no more than sixty degrees of natural motion of the knee. For example, the device can provide unloading from extension through at least five degrees and up to sixty degrees of flexion. Further, the first and second members can be configured to remain in contact throughout a full range flexion of a joint or can be arranged to be disengaged during portions of flexion.

A common joint procedure is joint replacement as previously described. The procedure of replacing a diseased joint includes resection of the surfaces of the joint and replacement with synthetic materials. To enable implantation of the load reducing system or knee unloading device without impacting the potential to complete subsequent procedures (e.g., joint replacement) the permanent fixation components in a preferred embodiment are positioned at a location that does not compromise the total joint zone. In other words, in a preferred embodiment, the entire load reducing system is extra-articular and entirely outside the joint capsule.

Many articulating joints are not simply pivot joints but involve complex multi-axis rotation and translation movements. To achieve its intended purpose, the load reducing device should accommodate these movements but also absorb and transfer energy during the required range of motion. To do so the device should be located at points on the bones selected to achieve the desired motion. The device joint locations may be finely adjusted within a defined region on the fixation component to further optimize the device joint location. In addition, the load reducing device may include one or more movable joint mechanisms (such as a universal joint) which accommodates the positional changes and therefore can increase the flexibility of fixation location for the members.

Methods of Implanting

The methods of implantation of a fully implanted or a partially external load reducing device can employ various degrees of non-invasive approaches for a given interventional procedure. Additional details and other embodiments of an load reducing system and methods of implantation are shown and described in U.S. patent application Ser. No. 11/775,139 entitled “Extra-Articular Implantable Mechanical Energy Absorbing Systems and Implantation Method” and a in U.S. patent application Ser. No. 12/113,160 entitled “Surgical Implantation Method and Device for an Extra-Articular Mechanical Energy Absorbing Apparatus,” which are both incorporated by reference in their entirety.

According to one implantation method, a pre-operative intervention session with the patient is conducted. By employing two-dimensional or three dimensional static or motion imaging techniques which are available, such as x-ray, MRI or CT scans, the anatomy of the interventional site can be examined and a dynamic assessment can be performed to map the members defining the particular joint. In a contemplated approach the medial proximal attachment site for the load reducing device can be located on a femur in a space bounded by the medial patellar retinaculum, the vastus medialis and the tibial collateral ligament. A distal attachment site can be located on the tibia in a space between the medical patellar retinaculum and the pes anserinus.

In one example of a method for implantation of the load reducing devices, spinal anesthesia or general anesthesia can be used and the implant site is accessed using minimally invasive techniques. In a preferred procedure to treat the knee joint, the Blumensaat's line of the femur bone is used as a landmark for locating the various components of the load reducing device as it has been found to provide a convenient initial position marker for ultimately achieving proper rotational positioning of the device. Other referencing points can additionally be used and of course are required when treating other joints.

In one approach, a first base is attached to the femur and a second base is attached to the tibia. Energy absorbing surfaces are provided at a junction between the bases. The surfaces allow multiple degrees of freedom between the first and second bases. The bases have a low-profile design and curved surfaces thereby minimizing the profile of the bases when mounted to the bone surface and enabling atraumatic motion of the adjoining soft tissues over the bases. The bases are secured to bone surfaces with one or more fastening members.

A number of embodiments are described above for adjusting the amount of load the load reducing device can manipulate to help reduce pain in a patient. These embodiments can be used in any load reducing system for use throughout the body but have clear applications to articulating body structures such as joints. Moreover, features and structures of certain of the disclosed embodiments can be incorporated into other disclosed embodiments by replacing structure or complementing structure. Certain of the embodiments include load reducing devices designed to minimize and complement the dampening effect and energy absorption provided by the anatomy of the body, such as that found at a body joint. It has been postulated that to minimize pain, load manipulation or absorption of 1-40% of forces on the natural joint, in varying degrees, may be necessary. Variable load manipulation or energy absorption in the range of 5-20% can be a target for certain applications. For one example of a knee joint, the load reducing device preferably is configured to support about 10-60 pounds, more preferably 20-50 pounds of spring force for load bypassing knee support providing therapeutic benefit for patients. Higher spring forces would provide greater reduction in joint load, may be used for heavier patients and may correlate to greater symptom (i.e., pain) relief.

In one embodiment, the load reducing system can be initially configured to unload, reduce or manipulate loads to a desired degree, and is later adjusted or altered as patient needs are better determined or change, i.e. as healing progresses.

It has been found that a medial compartment of a knee of an average person with osteoarthritis can benefit from an absorber set for compression between 1 mm and 10 mm, and preferably 3-6 mm with a spring or absorber element that accommodates a range from 20-60 pounds. In a preferred embodiment, the absorber is set for about 4 mm of such compression and a pre-determined load of about 30 pounds.

The terms “spring” and “absorber” are used throughout the description but it is contemplated to include other load reducing and compliant structures can be used to accomplish the functions of the invention

The various embodiments described above are provided by way of illustration only and should not be construed to limit the claimed invention. Those skilled in the art will readily recognize various modifications and changes that may be made to the claimed invention without following the example embodiments and applications illustrated and described herein, and without departing from the true spirit and scope of the claimed invention, which is set forth in the following claims. In that regard, various features from certain of the disclosed embodiments can be incorporated into other of the disclosed embodiments to provide desired structure.

What is claimed:

1. A method of surgical treatment of a joint to provide pain relief, the method comprising:
performing a surgical repair treatment on tissue within the joint capsule;
implanting a load reducing device at the joint and entirely outside of the joint capsule to reduce loads transmitted by the treated tissue to allow for the tissue within the joint capsule to heal; and
at least partially unloading the joint during healing of the surgical repair site.

2. The method of claim 1, wherein the surgical repair treatment is performed on articular cartilage.

3. The method of claim 2, wherein the surgical repair treatment includes an allograft or an autograft.

4. The method of claim 2, wherein the surgical repair treatment includes implantation of a biologic within the joint capsule.

5. The method of claim 4, wherein the surgical repair treatment includes implantation of stem cells.

6. The method of claim 2, wherein the surgical repair treatment includes meniscal repair.

7. The method of claim 1, wherein the surgical repair treatment is performed on bone.

8. The method of claim 7, wherein the surgical repair treatment is stem cell stimulation therapy involving microfracture or drilling of the bone.

9. The method of claim 1, wherein the surgical repair treatment involves cutting or repairing of joint cartilage.

10. The method of claim 1, wherein the joint is a knee joint.

11. The method of claim 1, wherein the surgical repair treatment is a failing or failed arthroplasty procedure.

12. The method of claim 1, wherein the surgical repair treatment and the implantation of the load reducing implantable device are performed during a same surgical procedure.

13. The method of claim 1, further comprising removing the load reducing device after a period of approximately 6 to approximately 24 months.

14. The method of claim 1, wherein load reducing device is entirely subcutaneous.

15. The method of claim 1, wherein the load reducing device is trancutaneous.

16. The method of claim 1, wherein the load reducing device comprises first and second bases affixed to first and second members of the joint, a resilient member spanning the joint, and at least one articulating member.

17. A method of surgical treatment of a joint, the method comprising:
performing autologous chondrocyte implantation;
implanting a load reducing device at the joint and entirely outside of the joint capsule to reduce load transmitted at the chondrocyte implantation site; and
allowing the new cartilage at the chondrocyte implantation site to mature for at least 6 months with reduced load bearing at the joint.

18. The method of claim 17, further comprising removing at least a part of the load reducing device after a period of approximately 6 to approximately 24 months.

19. The method of claim 17, further comprising deactivating the load reducing device after a period of approximately 6 to approximately 24 months.
